(19) United States
(12) Patent Application Publication
(10) Pub. No.: US 2005/0089509 A1
(43) Pub. Date: Apr. 28, 2005

(54) Treatment of Francisella infection with an IFN-gamma inducer and a chemotherapeutic agent

(75) Inventors: Bernard P. Arulanandam, San Antonio, TX (US); Karl E. Klose, San Antonio, TX (US); Judy M. Teale, San Antonio, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 10/939,950

(22) Filed: Sep. 13, 2004

Related U.S. Application Data
(60) Provisional application No. 60/502,482, filed on Sep. 12, 2003.

(51) Int. Cl. A61K 38/20; A61K 31/545; A61K 31/43; A61K 31/407

(52) U.S. Cl. 424/85.2; 514/192; 514/200; 514/210.09; 514/8

(57) ABSTRACT

The present invention concerns methods and compositions for treating or preventing Francisella infection in a subject comprising obtaining an inducer of IFN-γ, obtaining a chemotherapeutic agent, and administering the inducer of IFN-γ and the chemotherapeutic agent to the subject. The inducer of IFN-γ can be an IL-12 molecule, and the chemotherapeutic agent can be an antibiotic.

This application claims the benefit of U.S. Provisional Application Ser. No. 60/502,482, filed Sep. 12, 2003. The contents of the provisional application are incorporated by reference.

The government owns rights in the present invention pursuant to National Institutes of Health grants AR048973-02, SO6 GM008194-24, and WRCEU54 A1057156.

### BACKGROUND OF THE INVENTION

A. Field of the Invention

The present invention relates to the field of bacteriology. More particularly, it concerns methods and compositions for treating or preventing bacterial infections such as Francisella infection.

B. Description of Related Art

Bacterial infection, and the threat of such an infection, presents serious health cost and concerns in today's society. This is especially true where biological weapons can be used to spread and infect a large population of people with a given bacteria in a relatively short period of time.

The bacterium Francisella tularensis, for example, is the causative agent of tularemia (Tarnvk, 1989). F. tularensis has long been considered a potential biological weapon and has been actively studied as a potential germ warfare agent (Christopher et al., 1997; Dennis et al., 2001; Harris, 1992) because of its high rate of infectivity and capacity to cause illness and death in humans. (Dennis et al., 2001). Illnesses include pneumonia, pleuritis, and hilar lymphadenopathy. (Dennis et al., 2001).

F. tularensis can survive in several different environmental niches such as moist soil, water, hay, and animal carcasses. F. tularensis is an intracellular gram-negative bacterium that can be classified into several subspecies, including those most relevant to human disease: F. tularensis subsp. tularensis (type A) and F. tularensis subsp. holarctica (type B) (Ellis et al., 2002; Titball et al, 2003). An additional subspecies, novicida, shares biochemical and genetic similarities to F. tularensis and is highly virulent in mice (Forsman et al., 1994). Inhalation with as few as 10 organisms will cause disease that frequently is lethal and therefore requires prompt medical management.

Current treatment of tularemia in humans includes the use of aminoglycosides particularly streptomycin and gentamicin (Dennis et al., 2001; Enderlin et al., 1994; Evans et al., 1985; Mason et al., 1980; Maurin et al., 2000). Treatment regimens with these antibiotics involve prolonged daily therapy with relapses and failure rates up to 33% (Enderlin et al., 1994). Other classes of antibiotics including the fluoroquinolones, such as ciprofloxacin (Syrjala et al., 1991), have demonstrated some efficacy after prolonged treatment in mice (Russell et al., 1998) and humans (Johansson et al., 2000; Limaye and Hooper, 1999). Aerosol studies using F. tularensis SCHU 4 in monkeys showed that the animals had to be treated within 24 h of exposure with 13-daily consecutive doses of tetracycline (200 mg/intragastrically) to be effective (Sawyer et al., 1966). Delay of treatment resulted in febrile episodes and iiiness. In similar aerosol studies done on human volunteers, continual daily treatment with tetracycline for approximately 2 weeks was shown to be required to clear the infection (Sawyer et al., 1966). Factors that contribute to the poor performance of conventional antibiotics against intracellular bacteria include reduced cellular uptake (Johnson et al., 1980) and drug inactivation within subcellular compartments (Maurin and Raoult, 1996). A recent study by the Working Group on Civilian Biodefense (Dennis et al., 2001) reported the need for developing rapid post-exposure protection 4 against the illicit use of F. tularensis as a airborne bioweapon. Thus, any potential therapeutic application that can be safely administered with reduced dosage and length of treatment may provide a novel strategy to combat airborne pathogens.

The use of cytokines in combination with conventional antibiotics has shown promise against other intracellular bacteria (Ouadrhiri and Sibille, 2000). Interleukin-12 (IL-12) is a regulatory cytokine that activates T-helper (Th1) and NK cells to induce the production of interferon-γ (IFN-γ) (Trinchieri, 2003). The inventors (Arulanandam et al., 2001 a; Arulanandam et al., 1999; Arulanandam et al., 2001b) and others (Marinaro et al., 1999; Okada et al., 1997) have shown that the use of soluble IL-12 delivered intranasally (i.n.) as a potent and safe (Huber et al., 2003) vaccine adjuvant for stimulating protective mucosal immunity. The ability of IL-12 to induce efficient Th1 immune responses has been shown to be important in combinatorial immunotherapy (Stevens, 1998). IL-12 and antibiotics, such as clarithromycin and rifabutin, have been reported to promote clearance of Mycobacterium avium (Doherty and Sher, 1998). In addition, combination therapy using IL-12 and fluconazole was shown to be highly effective against cryptococcal infection (Clemons et al., 1994).

### SUMMARY OF THE INVENTION

The inventors have discovered a method and corresponding compositions for the treatment of bacterial infections such as Francisella infections. One aspect of this discovery is the inventors' identification of a synergistic effect between the administration of an inducer of IFN-γ and a chemotherapeutic agent to a subject that has a Accordingly, the present invention concerns therapeutic and preventative methods and compositions for treating or preventing Francisella infection in a subject comprising obtaining an inducer of IFN-γ, obtaining a chemotherapeutic agent, and administering the inducer of IFN-γ and the chemotherapeutic agent to the subject. The Francisella infection can be further defined as a Francisella tularensis infection. The Francisella tularensis infection can be further defined as a Francisella tularensis (subsp) novicida infection, a Francisella tularensis subsp. tularensis (Type A) infection, or a Francisella tularensis subsp. holarctica (Type B) infection.

The inducer of IFN-γ can, for example, increase the production or amount of IFN-γ in a given cell. This can be done, for example, by increasing the expression of the IFN-γ gene. The inducer of IFN-γ can also be a compound that activates macrophages, NK cells, and/or mediates antibody isotype switching to IgG2a. In particular embodiments, the inducer of IFN-γ is IL-12. Other non-limiting embodiments of IFN-γ inducers include TNF-α, IL-1, and IL-6. Those of skill in the art will be able to identify and test these and other relevant inducers of IFN-γ without undue experimentation using relevant resources and following standard identification and testing procedures.

The chemotherapeutic agents of the present invention can include, for example, synthetic and non synthetic compounds and molecules and their corresponding derivatives. Non-limiting examples include antimicrobial agents such as natural and synthetic antibiotics. In certain embodiments, the chemotherapeutic agent is an antibiotic, or a derivative of a given antibiotic. Non-limiting antibiotics that can be used with the present invention include Beta-lactams (penicillins and cephalosporins), semisynthetic penicillin, macrolides, sulfonamides, quinolones, aminoglycosides, fluoroquinolones, clavulanic, monobactams, carboxypenems, aminoglycosides, glycopeptides, lincomycins, macrolides, polypeptides, polyenes, rifamycins, tetracyclines, semisynthetic tetracycline, and chloramphenicol. These and other antibiotics discussed throughout this document and those known in the art are contemplated as being useful with the present invention. In certain embodiments, the antibiotic is doxycycline, streptomycin, gentamicin, or ciprofloxacin.

The chemotherapeutic agent and/or the IFN-γ inducer may be administered to a subject in a dose of about 0.1 to 10 mg/kg or μg/kg by weight of the subject. In other embodiments, the dosages can be from about 20, 30, 40, 50, 100, 200, 350, to about 500 mg/kg or μg/kg by weight of the subject. The chemotherapeutic agent and the IFN-γ inducer can be co-administered to a subject. In other aspects, the chemotherapeutic agent and the IFN-γ inducer can be administered simultaneously, at different times, in a single pharmaceutical composition, or in separate pharmaceutical compositions. In certain non-limiting embodiments, for example, IFN-γ inducer and the chemotherapeutic agent can be administered to the subject within about 1, 2, 3, 4, 5, 10, 15, 20, 30, 45, 50 to about 59 minutes of pre and/or post infection. In other non-limiting embodiments, the IFN-γ inducer and the chemotherapeutic agent can be administered to the subject within about 1, 2, 3, 4, 5, 6, 7, 8, 10, 12, 15, 20, 24, 36, 48, or 60 hours of pre or post infection. IFN-γ inducer and the chemotherapeutic agent can also be administered to a subject, for example, at about 8 hours after infection and at about 24 hours after infection, at about 24 hours after infection and at about 36 hours after infection, or at about 48 hours after infection and at about 60 hours after infection. The chemotherapeutic agent and the IFN-γ inducer can be administered intranasally, orally, or by injection. These and other aspects of the dosages of the chemotherapeutic agents and/or the IFN-γ inducers, the timing of administration, and the means of administration are discussed throughout this document and are contemplated as being useful with the present invention.

In another aspect of the present invention, there is provided a method of inducing IFN-γ production in a subject comprising obtaining an IFN-γ inducer, obtaining a chemotherapeutic agent, and administering the IFN-γ inducer and the chemotherapeutic agent to the subject. The method can be further defined as a method of treating or preventing Francisella infection in the subject. Also contemplated are compositions comprising an IFN-γ inducer and a chemotherapeutic agent in a pharmaceutically acceptable carrier, wherein said composition is adapted for intranasal administration. Non-limiting examples of IFN-γ inducers and chemotherapeutic agents are described throughout this document and are contemplated as being useful with this aspect of the present invention. Similarly, pharmaceutical compositions, the dosages, the timing of administration, and the means of administration of the chemotherapeutic agents and the IFN-γ inducers, are discussed throughout this document and are contemplated as being useful with this aspect of the present invention.

It is contemplated that any embodiment discussed in this document can be implemented with respect to any method or composition of the invention, and vice versa. Furthermore, compositions and kits of the invention can be used to achieve methods of the invention.

A “subject” refers to, for example, both mammals and non-mammals. Non-limiting examples include humans, mice, rabbits, pigs, cattle, sheep, goats dogs, cats, rats, rodents, or horses.

“Interleukin-12” and “IL-12” refer to interleukin 12 protein, its individual subunits, multimers of its individual subunits, functional fragments of IL-12, and functional equivalents and/or analogues of “interleukin-12” and “IL-12.” Functional fragments of IL-12 are fragments which, for example, when administered with a chemotherapeutic agent, treat or protect against Francisella infection. Functional fragments or equivalents of “interleukin-12” and “IL-12” include modified IL-12 protein such that the resulting IL-12 product has activity similar to the IL-12 described herein (e.g., the ability to when administered with a chemotherapeutic agent, treat or protect against Francisella infection). Functional equivalents or fragments of “interleukin-12” also include nucleic acid sequences (e.g., DNA, RNA) and portions thereof, which encode a protein or peptide having the IL-12 function or activity described herein (e.g., the ability to when administered with a chemotherapeutic agent, treat or protect against Francisella infection). In addition, the term includes a nucleotide sequence which through the degeneracy of the genetic code encodes a similar peptide gene product as IL-12 and has the IL-12 activity described herein. For example, a functional equivalent of “interleukin-12” and “IL-12” includes a nucleotide sequence which contains a “silent” codon substitution (e.g., substitution of one codon encoding an amino acid for another codon encoding the same amino acid) or an amino acid sequence which contains a “silent” amino acid substitution (e.g., substitution of one acidic amino acid for another acidic amino acid). An IL-12 molecule can also be a recombinant IL-12 molecule.

The use of the word “a” or “an” when used in conjunction with the term “comprising” in the claims and/or the specification may mean “one,” but it is also consistent with the meaning of “one or more,” “at least one,” and “one or more than one.”

The term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in the body of this document and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented in this document.

FIG. 1 The effects of intranasal IL-12 treatment on the course of pulmonary tularemia. 4 to 6 week old BALB/c mice (3 per group) were pre-treated i.n. with 100 ng of IL-12 in PBS-NMS or only PBS-NMS on days −1 and 0. All animals were challenged i.n. 4 h after the last treatment with 100 CFU of F. novicida. The animals were sacrificed 24 h and 72 h after infection and the organs removed, homogenized and plated. Colonies were enumerated after 24-36 hrs of incubation at 37° C. Results are shown as the mean±S.D. The differences in bacterial levels in the livers of IL-12-treated and PBS-treated mice at 72 h were significant at p<0.05.

FIG. 2 Prophylactic IL-12 treatment enhances protection against pulmonary Francisella challenge. BALB/c mice (5 mice/group) were pre-treated i.n. or i.p. with 100 ng of IL-12 in PBS-NMS on days −1 and 0. As controls, some animals were treated i.n. with PBS-NMS. All animals were challenged i.n. 4 h after the last treatment with 100 CFU of F. novicida. The animals were monitored daily for survival. The differences in time to death between i.n. IL-12-treated mice and PBS-treated mice were significant at p<0.005.

FIGS. 3A and 3(B) FIG. 3(A) Efficacy of combinatorial treatment with gentamicin and IL-12 against pulmonary tularemia. BALB/c animals (6/group) were challenged i.n. with 10₃ CFU of F. novicida in 25 μl of sterile PBS. 8 h+24 h after infection mice were treated i.n. with 100 μg of gentamicin+100 ng of soluble recombinant IL-12, 100 μg gentamicin alone, 100 ng of IL-12 alone, or PBS alone. FIG. 3(B) The animals were weighed and monitored daily for survival. Differences in survival between gentamicin/IL-12-treated mice and gentamicin alone were significant at p<0.001.

FIGS. 4A and 4(B) FIG. 4(A) The efficacy of delayed combinatorial treatment against pulmonary tularemia. BALB/c animals (6/group) were challenged i.n. with 10³ CFU of F. noicida. At varying intervals after challenge mice were treated i.n. with a combination of 100 μg of gentamicin+100 ng of soluble recombinant IL-12. As a control some animals were treated with PBS alone. FIG. 4(B) The animals were weighed and monitored daily for survival. Differences in survival between gentamicin/IL-12-treated mice at 8 h+24, 24 h+36 h and 48 h+60 h and PBS alone were significant at p<0.001.

FIGS. 5A and 5(B) FIG. 5(A) The effects of substituting IFN-γ for IL-12 in the combinatorial therapy against pulmonary tularemia. BALB/c animals (6/group) were challenged i.n. with 10³ CFU of F. novicida. 8 h+24 h after infection mice were treated i.n. with 100 μg of gentamicin+100 ng of soluble recombinant IFN-γ, or 100 μg gentamicin alone. FIG. 5(B) The animals were weighed and monitored daily for survival. Differences in survival between gentamicin+IFN-γ-treated mice and gentamicin alone were significant at p<0.001.

FIGS. 6A and 6(B) FIG. 6(A) The effects of IL-12 or IFN-γ on intracellular growth of Francisella. J774 cells (1×10⁵ cells/well) were infected at an MOI of 10:1 with F. novicida±5 ng/ml or 50 ng/ml of recombinant IL-12/IFN-γ for 1 h and then treated for and additional 1 h with medium containing gentamicin. Cells were then treated for 24 h±IL-12 or IFN-γ. The cells were washed with HBSS containing 0.1% gelatin and cell mixtures were lysed in 0.2% sodium deoxycholate and plated on TSA supplemented with 0.1% cysteine. Colonies were enumerated after 24-36 hrs of incubation at 37° C. FIG. 6(B) TNF-α levels in bacterial cultures treated with IL-12 or IFN-γ. TNF-α production in culture supernatants after 24 h of treatment was measured by ELISA. Results are shown as the mean±S.D. Differences in TNF-α levels between cells cultured with bacteria and IFN-γ and bacteria alone were significant at p<0.005.

FIG. 7 Model of Combinatorial Therapy Against Pulmonary Tularemia. IL-12 preferentially activates Th1 cells and NK cells to induce the production of IFN-γ, which directly enhances the anti-microbial affects of phagocytic cells such that bacteria that reside within cells is eradicated. Gentamicin will effectively inhibit bacterial replication outside cells. Alternatively, IL-12 and/or IFN-γ may directly act on macrophages and facilitate the entry of gentamicin into cells and thus eradicate the organisms.

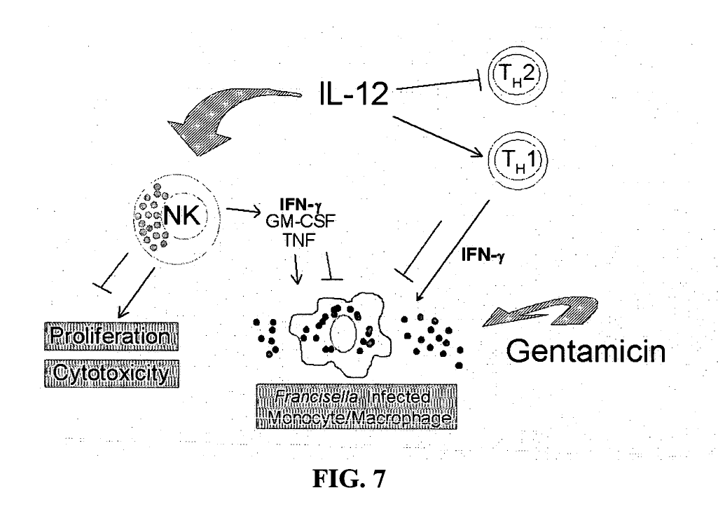

### DESCRIPTION OF ILLUSTRATIVE EMBODIMENTS

Management of intracellular bacterial infections using conventional antibiotic therapy can be difficult. Antibiotic treatment of such infections can result in high failure and relapse rates (Donowitz, 1994; Enderlin et al., 1994). Many of the obligate and facultative intracellular bacteria have evolved defense mechanisms that allow replication within phagocytic cells, resist intracellular killing and avoid immune surveillance (Fan et al., 2002; Ouadrhiri and Sibille, 2000; Zhong et al., 1999).

The respiratory tract and the lungs are a major portal of entry for inhalation and aerosol infections and serve as primary sites of infection before systemic spread. Delivery of bioweapons such F. tularensis and Bacillus anthracis by these routes have an additional dimension because of the extreme infectivity, ease of transmission and high fatality rate. This was particular evident with the illicit dissemination of inhalation B. anthracis and the resulting deaths from these incidents (Brookmeyer and Blades, 2002). All these obstacles pose significant challenges particularly with intracellular bacteria such as F. tularensis that are highly infectious and agents of biological warfare.

To this end, the present invention provides compositions and methods for treating bacterial infections such as F. tularensis bacterial infections by administering an inducer of IFN-γ alone, or in combination with, a chemotherapeutic agent. These and other aspects of the present invention are described in further detail throughout this document.

C. IFN-γ Inducers

IFN-γ inducers include compounds and molecules that can induce IFN-γ production in a subject. Non-limiting examples are provided throughout the body of this specification. An example of such an inducer is Interleukin-12 or IL-12 for short.

IL-12 is a heterodimeric cytokine that has a molecular weight of 75 kDa is composed of disulfide-bonded 40 kDa and 35 kDa subunits. It is produced by antigen presenting cells (“APC”) such as macrophages, and binds to receptors on activated T, B and NK cells. IL-12 has several effects including: (1) enhanced proliferation of T cells and NK cells; (2) increased cytolytic activities of T cells, NK cells, and macrophages; (3) induction of IFN-γ production and to a lesser extent, TNF-α and GM-CSF; and (4) activation of TH1 cells. IL-12 has been shown to be an important co-stimulator of proliferation in Th1 clones and leads to increased production of IgG2a antibodies in serum. Administration of IL-12 can also temporarily decrease production of IgG1 antibodies, indicating suppression of the Th2 response. The purification and cloning of IL-12 are disclosed in PCT publication nos. WO 92/05256 and WO 90/05147, and in European patent publication no. 322,827 (identified as “CLMF”).

IL-12 can be obtained from a variety of sources or synthesized by using known skills. For example, IL-12 can be purified (isolated, essentially pure) from natural sources (e.g., mammalian, such as human sources), produced by chemical synthesis, or produced by recombinant DNA techniques. In addition, IL-12 can be obtained from commercial sources.

D. Chemotherapeutic Agents

Chemotherapeutic agents include both synthetic and non synthetic compounds and molecules that can be used alone, or in combination with, IFN-γ inducers to prevent or treat a bacterial infection such as a Francisella infection. An example of a chemotherapeutic agent that can be used with the present invention is an antibiotic or a derivative of a given antibiotic. Such antibiotics may be any antibiotic, whether currently known or later discovered.

Antibiotics are any chemical of natural or synthetic origin that kill or inhibit the growth of other types cells. Many clinically-useful antibiotics are produced by microorganisms. Antibiotics are typically low molecular-weight (non-protein) molecules produced as secondary metabolites, mainly by microorganisms that live in the soil. Most of these microorganisms form some type of a spore or other dormant cell, and there is thought to be some relationship (besides temporal) between antibiotic production and the processes of sporulation. Among molds, the notable antibiotic producers are Penicillium and Cephalosporium, which are the main source of the beta-lactam antibiotics (penicillin and its relatives). In the Bacteria, the Actinomycetes, notably Streptomyces species, produce a variety of types of antibiotics including the aminoglycosides (e.g. streptomycin), macrolides (e.g. erythromycin), and the tetracyclines. Endospore-forming Bacillus species produce polypeptide antibiotics such as polymyxin and bacitracin.

Non-limiting antibiotics that can be used with the present invention include penicillins, cephalosporins, macrolides, sulfonamides, quinolones, aminoglycosides, beta-lactam antibiotics, and fluoroquinolones. In certain embodiments, the antibiotic is an aminoglycoside or fluoroquinolone. In more specific embodiments, the antibiotic is doxycycline, streptomycin, gentamicin, or ciprofloxacin. These and other antibiotics are disclosed in Table 1 below which includes a summary of the classes of certain antibiotics and their properties including their biological sources.

TABLE 1
Classes of antibiotics and their properties

| | | | Spectrum (effective | |
|---|---|---|---|---|
| Chemical class | Examples | Biological source | against) | Mode of action |
| Beta-lactams | Penicillin G, | Penicillium notatum and | Gram-positive bacteria | Inhibits steps in cell wall |
| (penicillins and | Cephalothin | Cephalosporium species | | (peptidoglycan) synthesis and murein |
| cephalosporins) | | | | assembly |
| Semisynthetic | Ampicillin, | | Gram-positive and Gram- | Inhibits steps in cell wall |
| penicillin | Amoxycillin | | negative bacteria | (peptidoglycan) synthesis and murein |
| | | | | assembly |
| Clavulanic Acid | Clavamox is | Streptomyces clavuligerus | Gram-positive and Gram- | Suicide inhibitor of beta-lactamases |
| | clavulanic acid | | negative bacteria | |
| | plus amoxycillin | | | |
| Monobactams | Aztreonam | Chromobacter violaceum | Gram-positive and Gram- | Inhibits steps in cell wall |
| | | | negative bacteria | (peptidoglycan) synthesis and murein |
| | | | | assembly |
| Carboxypenems | Imipenem | Streptomyces cattleya | Gram-positive and Gram- | Inhibits steps in cell wall |
| | | | negative bacteria | (peptidoglycan) synthesis and murein |
| | | | | assembly |
| Aminoglycosides | Streptomycin | Streptomyces griseus | Gram-positive and Gram- | Inhibit translation (protein synthesis) |
| | | | negative bacteria | |
| | Gentamicin | Micromonospora species | Gram-positive and Gram- | Inhibit translation (protein synthesis) |
| | | | negative bacteria esp. | |
| | | | Pseudomonas | |
| Glycopeptides | Vancomycin | Streptomyces orientales | Gram-positive bacteria, esp. | Inhibits steps in murein (peptidoglycan) |
| | | | Staphylococcus aureus | biosynthesis and assembly |
| Lincomycins | Clindamycin | Streptomyces lincolnensis | Gram-positive and Gram- | Inhibits translation (protein synthesis) |
| | | | negative bacteria esp. | |
| | | | anaerobic Bacteroides | |
| Macrolides | Erythromycin | Streptomyces erythreus | Gram-positive bacteria, | Inhibits translation (protein synthesis) |
| | | | Gram-negative bacteria not | |
| | | | enterics, | |
| | | | Neisseria, Legionella, | |
| | | | Mycoplasma | |
| Polypeptides | Polymyxin | Bacillus polymyxa | Gram-negative bacteria | Damages cytoplasmic membranes |
| | Bacitracin | Bacillus subtilis | Gram-positive bacteria | Inhibits steps in murein (peptidoglycan) |
| | | | | biosynthesis and assembly |
| Polyenes | Amphotericin | Streptomyces nodosus | Fungi | Inactivate membranes containing sterols |
| | Nystatin | Streptomyces noursei | Fungi (Candida) | Inactivate membranes containing sterols |
| Rifamycins | Rifampicin | Streptomyces mediterranei | Gram-positive and Gram- | Inhibits transcription (eubacterial RNA |
| | | | negative bacteria, | polymerase) |
| | | | Mycobacterium tuberculosis | |
| Tetracyclines | Tetracycline | Streptomycesspecies | Gram-positive and Gram- | Inhibit translation (protein synthesis) |
| | | | negative bacteria, | |
| | | | Rickettsias | |
| Semisynthetic | Doxycycline | | Gram-positive and Gram- | Inhibit translation (protein synthesis) |
| tetracycline | | | negative bacteria, | |
| | | | Rickettsias Ehrlichia, | |
| | | | Borellia | |
| Chloramphenicol | Chloramphenicol | Streptomyces venezuelae | Gram-positive and Gram- | Inhibits translation (protein synthesis) |
| | | | negative bacteria | |

E. Combinational Strategies

In order to increase the effectiveness of treating a bacterial infection such as a Francisella infection, the inventors contemplate a combinational strategy of administering an IFN-γ inducer along with a chemotherapeutic agent to a subject. In one example, it is contemplated that a composition may include both an IFN-γ inducer and a chemotherapeutic agent such as an antibiotic.

In another example, the administration of an IFN-γ inducer can proceed or follow the administration of a chemotherapeutic agent by intervals ranging from minutes to weeks. It is contemplated that one may administer both modalities within about 1-24, 6-12, or 4-8 hours from each other, and any range derivable therein. In other aspects, it is contemplated that one may administer both modalities within about 0-60, 5-50, 10-40, or 20-30 minutes from each other, and any ranger derivable therein. In some situations, it may be desirable to extend the time period for treatment significantly, where several days (2, 3, 4, 5, 6 or 7) to several weeks (1, 2, 3, 4, 5, 6, 7 or 8) lapse between the respective administrations.

Various combinations may be employed where a compositions including an IFN-γ inducer is “A” and a chemotherapeutic agent or other secondary agent, is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

F. Compositions

A person of ordinary skill would recognize that the compositions of the present invention can include a varying range of concentrations of an IFN-γ inducer(s) and/or a chemotherapeutic agent(s). In certain non-limiting embodiments, the compositions may comprise in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 99% or more of at least one IFN-γ inducer(s) and/or a chemotherapeutic agent(s). A person of ordinary skill in the art would understand that the concentrations of an IFN-γ inducer(s) and/or a chemotherapeutic agent(s) can vary depending on the addition, substitution, and/or subtraction of additional materials.

The compositions of the present invention may also include additional antibacterial, antiviral, or antifungal agents, including but not limited to parabens (e.g., methylparabens, propylparabens), chlorobutanol, phenol, sorbic acid, thimerosal, or combinations thereof.

In certain embodiments, the compositions of the present invention will be formulated as a pharmaceutical composition. The phrases “pharmaceutical or pharmacologically acceptable” refers to molecular entities and compositions that do not produce an adverse, allergic or other untoward reaction when administered to an animal, such as, for example, a human. The preparation of a pharmaceutical composition including an IFN-γ inducer(s) and/or a chemotherapeutic agent(s) is known to those of skill in the art in light of the present disclosure, and as exemplified by Remington's Pharmaceutical Sciences, 18th Ed. Mack Printing Company, 1990. For animal (e.g., human) administration, it will be understood that preparations should meet sterility, pyrogenicity, general safety and purity standards as required by FDA Office of Biological Standards.

“Therapeutically effective amounts” are those amounts effective to produce beneficial results in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

An effective amount of the pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease.

A “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combination s thereof, as would be known to one of ordinary skill in the art (Remington's, 1990). Except insofar as any conventional carrier is incompatible with the active ingredient, its use in the therapeutic or pharmaceutical compositions is contemplated.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, for example, pharmaceutical compositions may comprise at least about 0.1%, 0.2%, 0.3%, 0.4%, 0.5%, 0.6%, 0.7%, 0.8%, 0.9%, 1%, 1.1%. 1.2%, 1.3%, 1.4%, 1.5%, 1.6%, 1.7%, 1.8%, or 1.9% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Alternatively, a patient may be given 1×10⁻⁵, 10⁻⁶, 10⁻⁶, 10⁻⁷, 10⁻⁸, 10⁻⁹, 10⁻¹⁰, 10⁻¹¹, 10⁻¹² M of a substance (or any range derivable therein), such as an IFN-γ inducer(s) and/or a chemotherapeutic agent(s) in a volume of 0.1 to 1, 10 to 1, 100 to 1, 1 ml, 5 ml, 10 ml, 20 ml, 25 ml, 50 ml, 100 ml, 200 ml, 300 ml, 400 ml, 500 ml, or more (or any range derivable therein).

The compositions of the present invention may comprise different types of carriers depending on whether it is to be administered in solid, liquid or aerosol form, and whether it need to be sterile for such routes of administration injection.

The compositions may be formulated into a composition in a free base, neutral, or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments, the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

G. Routes of Administration

Compositions of the present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrauterinely, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (Remington's, 1990).

Inhalation of as few as 10 organisms of F. tularensis can cause disease and significant mortality in humans. Because of this, a person of ordinary skill will recognize the value of intranasal and/or intrapulmonary administration of an IFN-γ inducer(s) and/or a chemotherapeutic agent(s) in treating this type of bacterial infection. The inventors have performed significant previous work relating to intranasal administration of active compounds, and, in particular IL-12. Those of ordinary skill will, in view of this specification and the inventor's past work, will be able to prepare and administer an IFN-γ inducer(s) and/or a chemotherapeutic agent(s) via intranasal routes (Arulanandam, 2003). See also, co-pending U.S. patent application Ser. No. 09/035,188, “Enhancement of Immunity by Intranasal Inoculation of IL-12,” by Metzger and Arulanandam.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials

Bacteria: F. tularensis subsp. novicida was kindly provided by Dr. Francis Nano from the Department of Biochemistry and Microbiology, University of Victoria, Canada. Bacteria were grown at 37° C. in Typticase Soy broth (TSB) supplemented with 0.1% cysteine. Aliquots of bacteria were stored at −70° C. in TSB containing 80% glycerol.

Mice: Six-to eight-week old female BALB/c mice were obtained from the National Cancer Institute (Bethesda, Md.). IFN-γ deficient (IFN-γ^{-/-}) and wild type (IFN-γ^{+/+}) BALB/c mice and NK cell deficient (C57BL/6J-Lyst^{bg-j}) and wild type (C56BL/6J) mice were obtained from the Jackson Laboratories (Bar Harbour, Me.). Mice were housed in the animal facility at the University of Texas at San Antonio and provided food and water ad libitum. All animal care and experimental procedures were performed in compliance with the Institutional Animal Care and Use Committee (IACUC) guidelines.

### Example 2

### Intranasal IL-12 Treatment, Bacterial Load Assessment, and Survival Studies

For intranasal (“i.n.”) treatment, mice were first anesthetized with 3% Isofluorane using a rodent anesthesia system (Harvard Apparatus, Holliston, Mass.) (Huber et al., 2003). Anesthetized animals were treated i.n. on days −1 and 0 with 100 ng of IL-12 in PBS containing 1% normal BALB/c mouse serum (PBS-NMS) or in the case of control mice, with PBS-NMS alone. The i.n. route of IL-12 delivery was chosen based on preliminary experiments comparing intraperitoneal (i.p.) and i.n. treatments. The i.n. route was found to be the most efficacious with no toxicity observed. This is in agreement with the previous findings on the feasibility of using IL-12 as a mucosal adjuvant (Arulanandam et al., 2001a; Arulanandam et al., 1999; Arulanandam et al., 2001b). All animals were then challenged i.n. 4 h after the final treatment with 100 CFU (10 LD₅₀) of F. novicida. The actual inoculum concentration of bacteria for each experiment was determined by serial dilution and plating on TSA supplemented with 0.1% cysteine. Bacterial load was assessed in groups of infected animals by removal of target organs 24 h and 72 h after pulmonary challenge. Organs were homogenized with an electric stirrer (Arrow Junior, Kimble/Kontes, Vineland, N.J.). The homogenates were serially diluted and plated on TSA supplemented with 0.1% cysteine and incubated for 18-24 h at 37° C. for bacterial enumeration. For survival studies, mice were treated either i.n. or i.p. with 100 ng of IL-12 in PBS-NMS on days on days −1 and 0 and challenged 4 h later with 100 CFU of F. novicida. All animals were monitored daily for morbidity and mortality.

### Example 3

### Pulmonary Francisella Infection and IL-12/Gentamicin Treatment

For intranasal challenge and treatment, mice were first anesthetized as described. Animals were immediately challenged i.n. with 1000 CFU of F. novicida in 25 μl of sterile PBS. The larger challenge dose (1000 CFU or approximately 100 LD₅₀) was selected for the treatment studies to better assess the efficacy of combinatorial therapy. Animals were treated i.n. at 8 h+24 h after challenge with 100 μg of gentamicin (Invitrogen, Carlsbad Calif.), or a combination of gentamicin plus 100 ng of recombinant murine IL-12 (R&D Systems, Minneapolis, Minn.) in PBS-NMS. Some groups of mice were treated with IL-12 or PBS-NMS alone. In some experiments, the inventors also examined the effects of delaying treatment against pneumonic tularemia. All mice were monitored daily.

### Example 4

### Phagocytosis Assay

The effects of IL-12 and IFN-γ on intracellular growth of Francisella was studied using murine macrophages (J774 cells; ATCC, Manassas, Va.). J774 7 cells (1×10⁵ cells/well) were incubated in microtiter plates and infected at a multiplicity of infection (MOI) of 10:1 with F. novicida±5 ng/ml or 50 ng/ml of recombinant IL-12 or IFN-γ (R&D Systems) for 1 h. Cultures were then treated for an additional 1 h with medium containing gentamicin (10 μg/ml) to eliminate extracellular bacteria. Cells were then treated for 24 h±IL-12 or IFN-γ. The cells were subsequently washed with HBSS containing 0.1% gelatin and cell mixtures were lysed in 0.2% sodium deoxycholate (Sigma) and plated on TSA supplemented with 0.1% cysteine. Colonies were enumerated after 24-36 hrs of incubation at 37° C. TNF-α secretion by activated macrophages was measured by ELISA as described (Arulanandam et al., 2001).

### Example 5

### Statistical Analysis

Survival data were analyzed by the Mann-Whitney rank sum test and the bacterial load and in vitro studies evaluated by Student's t-test using the statistical software program SigmaStat. The data are presented as mean±standard deviation.

### Example 6

### Synergistic Effect of Intranasal IL-12/Antibiotic Treatment Against Pulmonary Francisella Infection

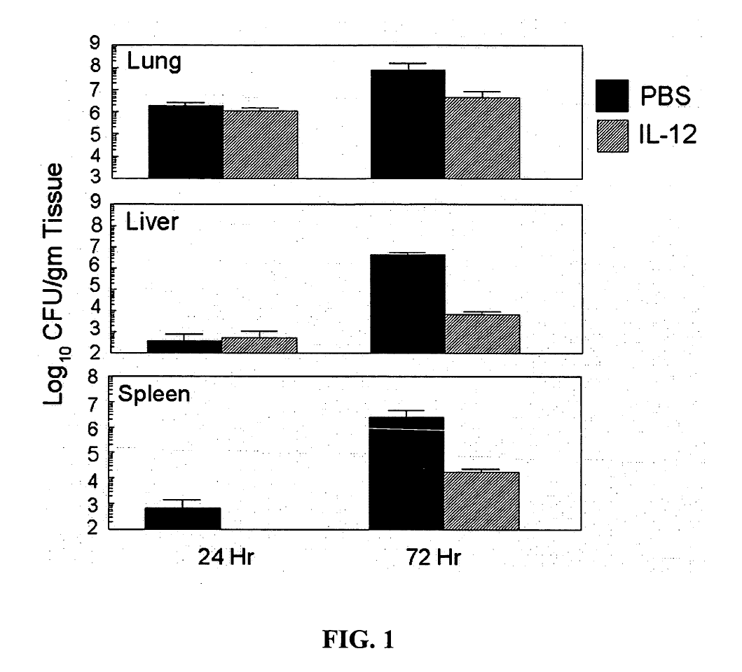

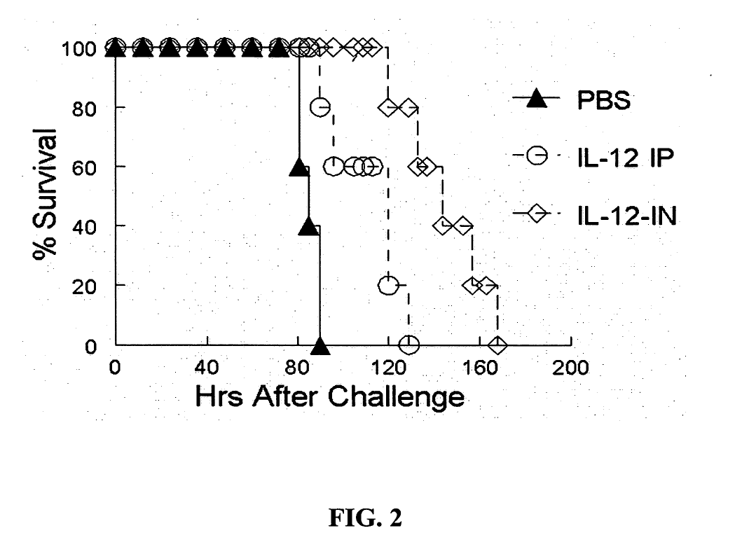

To directly assess whether IL-12 treatment can mediate resistance to pulmonary Francisella infection, BALB/c mice were pre-treated (on days −1 and 0) with 100 ng of IL-12 or PBS-NMS i.n. and subsequently challenged by the same route with 102 CFU of F. novicida. The animals were sacrificed 24 h and 72 h after challenge and the bacterial load assessed in various target organs. Within 24 h of pulmonary Francisella infection, there were similar levels of bacteria recovered from the lungs and liver of both sets of animals (FIG. 1). However, there were no bacteria recovered at 24 h from the spleens of the IL-12 treated animals in contrast to controls. By 72 h after i.n. challenge, IL-12 treated animals displayed a marked reduction of bacteria in the liver and spleen compared to animals treated with PBS-NMS. To determine the effects of prophylactic treatment with IL-12 on survival, groups of mice were pretreated with IL-12 i.n. or i.p. and subsequently challenged with 10² CFU of F. novicida. It was found, that IL-12 treatment i.n. significantly increased the time to death compared to animals that were pre-treated with IL-12 i.p. or animals receiving vehicle alone (FIG. 2).

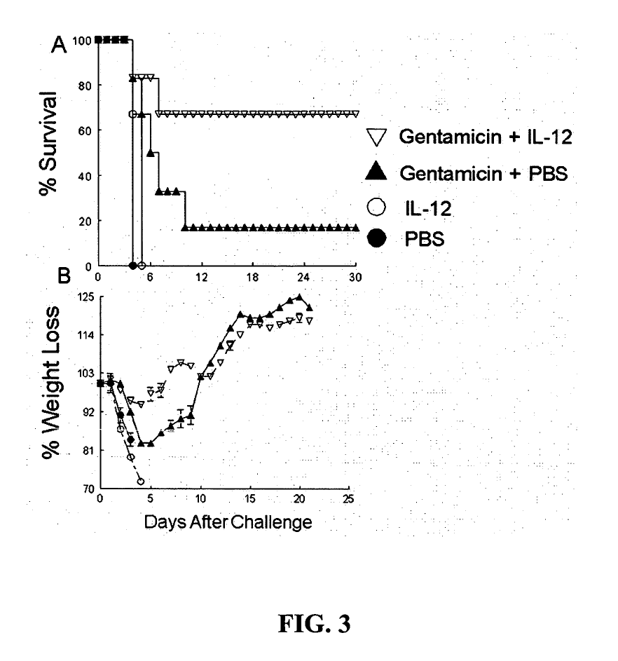

To determine the therapeutic efficacy of the combinatorial approach against Francisella, the inventors utilized IL-12 and gentamicin. Gentamicin is a conventional antibiotic that is used to treat individuals with acquired tularemia (Dennis et al., 2001). BALB/c mice were challenged i.n. with 10³ CFU of F. novicida. At 8 h+24 h after pulmonary challenge, mice were treated with combinations of gentamicin (100 μg) and IL-12 (100 ng), gentamicin alone, IL-12 alone or PBSNMS. This therapeutic treatment regimen revealed that the combinatorial effects of IL-12 and gentamicin were surprisingly and unexpectedly highly efficacious resulting in about 70% survival against this highly infectious organism (FIG. 3A). This also was evident by the gradual increase in body weight of these treated animals (FIG. 3B). In contrast to the combinatorial treatment, animals treated with gentamicin alone displayed only 17% survival, and animals receiving either IL-12 or PBS-NMS alone succumbed to the infection by day 6. The clearance of the bacterial infection also was evident by histological analyses, whereby the lungs of animals treated with combinatorial therapy 30 days after the challenge looked markedly healthy with minimal presence of organisms (data not shown).

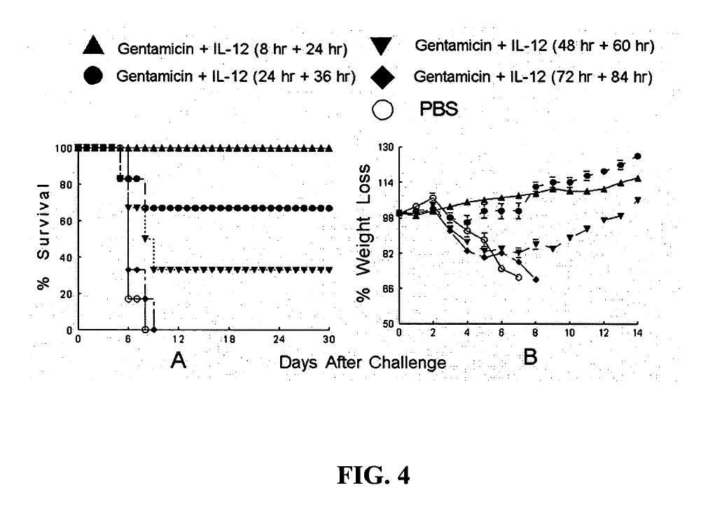

To determine the efficacy of this combined treatment delivered at later times during an ongoing infection, the inventors delayed the combinatorial treatment regimen over a span of 4 days. As shown in FIG. 4A, delayed treatment resulted in reduction of survival rates that correlated with the length of time between challenge and initiation of therapy. Animals treated at 8 h+24 h after challenge (10³ CFU) were highly protected as expected with minimal loss of body weight (FIG. 4b). When the treatment was delayed to 24 h+36 h post infection, the survival rate dropped to 66%, but was still statistically significant compared to untreated controls p<0.05). Further delaying the treatment to 48 h+60 h post challenge resulted in about 40% survival, whereas delaying treatment to 72 h+84 h after challenge resulted in death of all of the animals.

### Example 7

### The Effects of Combinatorial 11-12/Gentamicin are Dependent on IFN-γ

IL-12 administered i.n. or parenterally has profound regulatory effects on the immune system through its ability to preferentially activate Th1 and NK cells and induce IFN-γ production (Trinchieri, 2003). To assess the contribution of IFN-γ in mediating the therapeutic effects of the combined treatment, IFN-γ^{-/-} and corresponding wild-type mice were infected with F. novicida and treated 8 h+24 h later with gentamicin plus IL-12. All of the IFN-γ^{-/-} animals succumbed to the infection by day 5 in contrast to 80% protection through 30 days in the IFN-γ^{+/+} animals (data not shown). Because NK cells serve as a prime target for IL-12 and play an important role in innate immunity, the inventors determined the role of these cells in mediating the protective effects of this treatment. Mice defective in NK cell activity (C57BL/6J^{bg}) and corresponding wild type (C57BL/6J) animals were infected and treated as described. Whereas 50% of NK cell deficient animals survived the disease, all the wild-type animals were fully protected (data not shown). There was only a transient loss of weight seen in treated wild-type animals, whereas the NK cell deficient mice displayed enhanced weight loss and only gradually regained initial body weight.

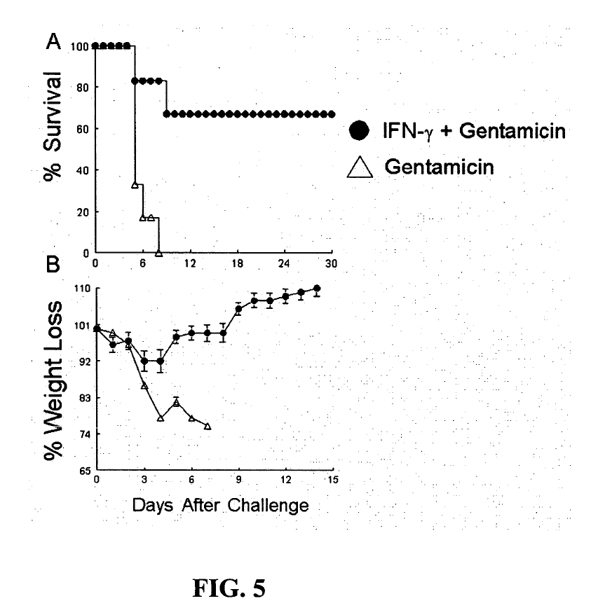

Because the effect of IL-12 on drug-induced bacterial clearance is dependent on IFN-γ, the inventors investigated whether therapeutic administration of IFN-γ would directly augment the effects of gentamicin. Mice infected i.n. with F. novicida were treated at 8 h+24 h after challenge with a combination of either 100 ng murine IFN-γ and gentamicin or gentamicin alone. As shown in FIG. 5, IFN-γ effectively substituted for the activity of IL-12 and promoted survival against F. novicida. In contrast, i.n. treatment with gentamicin alone was ineffective in controlling the disease. These results suggest that the combinatorial effects of IL-12/gentamicin treatment were highly dependent on IFN-γ production.

### Example 8

### IFN-γ Treatment Effectively Inhibits Intracellular Replication of Francisella

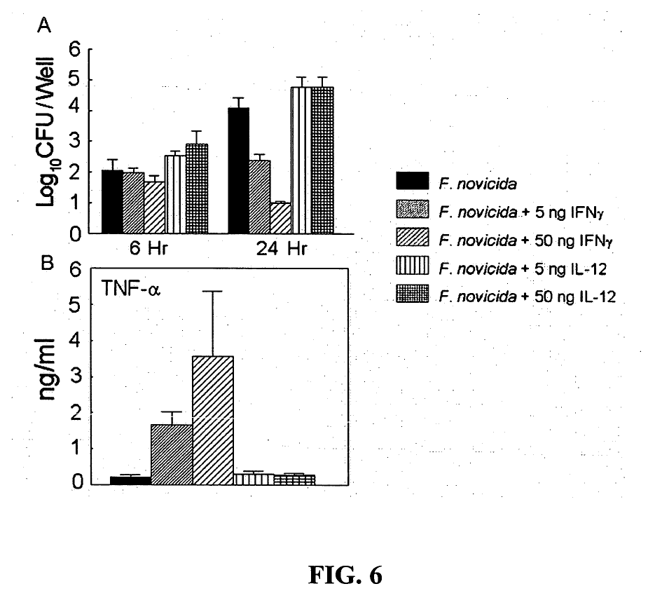

To determine the effects of IL-12/IFN-γ treatment on bacterial replication, J774 macrophages were infected with F. novicida in the presence or absence of recombinant IL-12 or IFN-γ for 24 hr. The cells were lysed and homogenates plated to determine bacterial growth. As shown in FIG. 6A, F. novicida replicates significantly in macrophages over a span of 24 h. Treatment of cells with either 5 ng or 50 ng of IL-12 did not inhibit bacterial replication. However, macrophages treated with either 5 ng or 50 ng of IFN-γ during the 24 h period markedly inhibited replication of bacteria in a concentration dependent-manner. Upon activation, murine macrophages are known to produce TNF-α which plays a pivotal role in the antimicrobial effects of these cells (Havell, 1992). Whereas very little TNF-α was detected in macrophages exposed to bacteria±IL-12, cells incubated with bacteria in the presence of IFN-γ induced significant amounts of TNF-α (9-fold increase with 5 ng IFN-γ and 18-fold increase with 50 ng IFN-γ) at 24 h (FIG. 6B). Enhanced TNF-α secretion by IFN-γ-stimulated cells may be involved in the significant inhibition of F. novicida replication.

### Example 9

### Discussion

Using an i.n. treatment approach to directly target the respiratory compartment, the inventors have shown the effectiveness of combinatorial therapy using a chemotherapeutic agent (gentamicin) plus an IFN-γ inducer (IL-12) to treat pulmonary tularemia. This treatment regimen administered twice i.e., (at 8 h+24 h) after challenge significantly protected animals from pulmonary tularemia compared with animals treated either with the drug or IL-12 alone. Delaying the combinatorial therapy to 24 h+36 h was still highly effective against the advanced form of the pulmonary disease as seen by 67% survival, while further delaying this therapy to 48 h+60 h resulted in 33% survival.

The results show that the effects of the combinatorial approach in treating pulmonary tularemia is primarily mediated by IFN-γ and that NK cells may contribute to clearance of this pulmonary infection. IFN-γ has a variety of immunoregulatory functions, which include induction of Th1 cell differentiation and activation of NK cells (Trinchieri, 2003). The greater reduction after IL-12 treatment of bacterial loads in the liver and spleen compared to the lungs, the primary site of infection, may be attributed to the increased numbers of NK and NK T cells in these organs that can be directly activated by IL-12 to enhance cytolytic activity and killing of infected cells (Matsushita et al., 1999). IFN-γ also has been shown to directly activate macrophage activity and killing (Munder et al., 1998). The failure of the combinatorial therapy in infected IFN-γ deficient animals supports the pivotal role of IFN-γ in activating innate defenses. In addition, IFN-γ-activated macrophages very efficiently mediated intracellular killing of Francisella in vitro compared to cells treated with IL-12. This enhanced killing may be related to the augmented induction of TNF-α seen after IFN-γ treatment of the macrophages. These results are in agreement with Fortier et al. (1992), who have shown that TNF-α may act as an autocrine signal to amplify IFN-γ-induced production of NO to inhibit growth of Francisella. Although IL-12 plays a major role in regulating IFN-γ mediated clearance of Francisella infection, there may be mechanisms independent of IFN-γ that involve the IL-12 p40 subunit (Elkins et al., 2002) alone or in combination with cytokines such as IL-23 (van de Vosse et al., 2003).

The inventors have also shown that direct administration of IFN-γ synergies with gentamicin to promote bacterial clearance. IL-12 may be the more appropriate cytokine for combinatorial therapy for several reasons. IL-12 has a longer sustainable half-life than IFN-γ in vivo and is produced up-stream of IFN-γ and should induce greater amounts of the latter cytokine than the injected dose (Gately et al., 1994). Additionally, i.n. treatment with IL-12 is associated with very little toxicity as demonstrated by the recent report (Huber et al., 2003) that shows IL-12 delivered i.n. induces less systemic IFN-γ production and fewer pathological changes. The inventors are aware that i.n. pulmonary challenges tend to deposit in the respiratory mucosa, whereas aerosol delivery results in dispersion of infectious inoculum into the alveoli (McMurray, 2001).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. patent Ser. No. 09/035,188
Arulanandam and Metzger, Vaccine. 17:252-260, 1999.
Arulanandam et al., Eur. J. Immunol., 29:256-264, 1999.
Arulanandam et al., J. Infect. Dis, 180:940-949, 1999.
Arulanandam et al., Infect. Immun., 69:6718-6724, 2001 a.
Arulanandam et al., J. Immunol., 166:226-231, 2001b.
Arulanandam et al., Int. Immunopharm., 3:801-809, 2003
Brookmeyer and Blades, Science, 295:1861, 2002.
Christopher et al., JAMA, 278:412-417, 1997.
Clemons et al., Antimicrob. Agents Chemother., 38:460-464, 1994.
Dennis et al., JAMA, 285:2763-2773, 2001.
Doherty and Sher, J. Immunol., 160:5428-5435, 1998.
Donowitz, Clin. Infect. Dis., 19:926-930, 1994.
Elkins et al., Infect. Immun., 70:1936-1948, 2002.
Ellis, Tularemia. Clin. Microbiol. Rev., 15:631-646, 2002.
Enderlin et al., Clin. Infect. Dis., 19:42-47, 1994.
European Pat. Appln. 322,827
Evans et al., Medicine, 64:251-269, 1985.
Fan et al., Infect. Immun., 70:345-349, 2002.
Forsman et al., Int. J. Syst. Bacteriol., 44:38-46, 1994.
Fortier et al., Infect. Immun., 60:817-825, 1992.
Gately et al., Ther. Immunol., 1:187-196, 1994.
Harris, Ann. N.Y. Acad. Sci., 666:21-52, 1992.
Havell, Immunol. Ser., 56:341-363, 1992.
Huber et al., Int. Immunopharmacol., 3:801-809, 2003.
Johansson et al., Pediatr. Infect. Dis. J., 19:449-453, 2000.
Johnson et al., J. Lab. Clin. Med., 95:429-439, 1980.
Limaye and Hooper, Clin. Infect. Dis., 29:922-924, 1999.
Marinaro et al., J. Immunol., 162:114-121, 1999.
Mason et al., Am. Rev. Respir. Dis., 121:39-45, 1980.
Matsushita et al., Int. Immunol., 11:657-665, 1999.
Maurin et al., Antimicrob. Agents Chemother., 44:3428-3431, 2000.
Maurin and Raoult, Trends Mol. Med., 7:135-137, 1996.
Munder et al., J. Exp. Med., 187:2103-2108, 1998.
PCT Appln. WO 92/05256
PCT Appln. WO 90/05147
Okada et al., J. Immunol., 159:3638-3647, 1997.
Ouadrhiri and Sibille, Curr. Opin. Infect. Dis., 13:233-240, 2000.
Remington's Pharmaceutical Sciences, 18th Ed. Mack Printing Company, 1289-1329, 1990.
Russell et al., J. Antimicrob. Chemother., 41:461-465, 1998.
Sawyer et al., Bacteriol. Rev., 30:542-550, 1966.
Stevens, Clin. Infect. Dis., 26:1266-1269, 1998.
Syrjala et al., Eur. J. Clin. Microbiol. Infect. Dis., 10:68-70, 1991.
Tarnvik, Rev. Infect. Dis., 11:440-451, 1989.
Titball et al., Trends Microbiol., 11:118-123, 2003.
Trinchieri, Nat. Rev. Immunol., 3:133-146, 2003.
Van de Vosse et al., Immunogenetics, 54:817-829, 2003.
Zhong et al., J. Exp. Med., 189:1931-1938, 1999.

1. A method of treating or preventing Francisella infection in a subject comprising:
(a) obtaining an IL-12 molecule;
(b) obtaining a chemotherapeutic agent; and
(c) administering the IL-12 molecule and the chemotherapeutic agent to the subject.

2. The method of claim 1, wherein the Francisella infection is further defined as Francisella tularensis infection.

3. The method of claim 2, wherein the Francisella tularensis infection is further defined as a Francisella tularensis (subsp) novicida infection.

4. The method of claim 2, wherein the Francisella tularensis infection is further defined as Francisella tularensis subsp. tularensis infection.

5. The method of claim 2, wherein the Francisella tularensis infection is further defined as Francisella tularensis subsp. holarctica infection.

6. The method of claim 1, wherein the IL-12 molecule is a recombinant IL-12 molecule.

7. The method of claim 1, wherein the IL-12 molecule is administered in a dose of 0.5 to 150 μg/kg weight of the subject.

8. The method of claim 1, wherein the chemotherapeutic agent is an antibiotic.

9. The method of claim 1, wherein the chemotherapeutic agent is administered in a dose of 1 to 10 mg/kg weight of the subject.

10. The method of claim 8, wherein the antibiotic is an aminoglycoside or fluoroquinolone.

11. The method of claim 8, wherein the antibiotic is doxycycline, streptomycin, gentamicin, or ciprofloxacin.

12. The method of claim 11, wherein the antibiotic is gentamicin.

13. The method of claim 12, wherein the gentamicin is administered in a dose of 0.1 to 250 mg/kg weight of the subject.

14. The method of claim 1, wherein the IL-12 and chemotherapeutic are co-administered.

15. The method of claim 1, wherein the IL-12 and chemotherapeutic are administered simultaneously.

16. The method of claim 1, wherein the IL-12 and chemotherapeutic are administered at different times.

17. The method of claim 1, wherein the IL-12 and chemotherapeutic are administered in a single pharmaceutical composition.

18. The method of claim 1, wherein the IL-12 and chemotherapeutic are administered in a separate pharmaceutical compositions.

19. The method of claim 1, wherein the IL-12 and chemotherapeutic are administered by intranasal introduction.

20. The method of claim 1, wherein the IL-12 and chemotherapeutic are administered by injection.

21. The method of claim 1, wherein the subject is a mouse.

22. The method of claim 1, wherein the subject is a human.

23. The method of claim 1, wherein the IL-12 molecule and the chemotherapeutic agent are administered to the subject within about 8 hours of infection.

24. The method of claim 1, wherein the IL-12 molecule and the chemotherapeutic agent are administered to the subject within about 24 hours of infection.

25. The method of claim 1, wherein the IL-12 molecule and the chemotherapeutic agent are administered to the subject at about 8 hours after infection and at about 24 hours after infection.

26. The method of claim 1, wherein the IL-12 molecule and the chemotherapeutic agent are administered to the subject within about 36 hours of infection.

27. The method of claim 1, wherein the IL-12 molecule and the chemotherapeutic agent are administered to the subject at about 24 hours after infection and at about 36 hours after infection.

28. The method of claim 1, wherein the IL-12 molecule and the chemotherapeutic agent are administered to the subject within about 48 hours of infection.

29. The method of claim 1, wherein the IL-12 molecule and the chemotherapeutic agent are administered to the subject within about 60 hours of infection.

30. The method of claim 1, wherein the IL-12 molecule and the chemotherapeutic agent are administered to the subject at about 48 hours after infection and at about 60 hours after infection.

31. A method of treating or preventing Francisella infection in a subject comprising:
(a) obtaining an inducer of IFN-γ;
(b) obtaining a chemotherapeutic agent; and
(c) administering the inducer of IFN-γ and the chemotherapeutic agent to the subject.

32. The method of claim 31, wherein the inducer of IFN-γ is a compound that activates macrophages and NK cells and mediates antibody isotype switching to IgG2a.

33. The method of claim 32, wherein the inducer of IFN-γ is IL-12.

34. The method of claim 31, wherein the chemotherapeutic agent is an antibiotic.

35. The method of claim 34, wherein the antibiotic is an aminoglycoside or fluoroquinolone.

36. The method of claim 34, wherein the antibiotic is doxycycline, streptomycin, gentamicin, or ciprofloxacin.

37. The method of claim 36, wherein the antibiotic is gentamicin.

38. A composition comprising an IL-12 molecule and a chemotherapeutic agent in a pharmaceutically acceptable carrier, wherein said composition is adapted for intranasal administration.

39. The composition of claim 38, wherein the chemotherapeutic agent is an antibiotic.

40. The composition of claim 39, wherein the antibiotic is gentamicin.
